(19) United States
(12) Patent Application Publication
(10) Pub. No.: US 2022/0169971 A1
(43) Pub. Date: Jun. 2, 2022

(54) HYDROGEL-BASED 3D CELL TRAINING BIOREACTOR

(71) Applicant: The Chinese University of Hong Kong, Hong Kong (CN)

(72) Inventors: Yangzi JIANG, Hong Kong (CN); Jing SUN, Hong Kong (CN)

(73) Assignee: The Chinese University of Hong Kong, Hong Kong (CN)

(21) Appl. No.: 17/533,991

(22) Filed: Nov. 23, 2021

Related U.S. Application Data
(60) Provisional application No. 63/118,754, filed on Nov. 27, 2020.

(51) Int. Cl. C12M 1/42; C12N 5/077; C12M 1/36; A61F 2/38

(52) U.S. Cl. CPC C12M 35/06; C12N 5/0655; C12M 41/48; C12N 2533/30; C12N 2529/00; C12N 2533/54; A61F 2/3872

(57) ABSTRACT

The subject invention pertains to a device and methods for inducing tensile strain on a hydrogel and/or hydrogel-encapsulated cells and/or tissues. The device includes a hydrogel-based mold for cell culture and a magnet-combined rail slider for cyclic tensile stretch. The resulting hydrogel-based device provides controllable tensile strain to cells and/tissues, from which cells and/or tissues can be encapsulated in hydrogels and strain can be applied cyclically.

### CROSS-REFERENCE TO RELATED APPLICATION(S)

This application claims the benefit of U.S. Provisional Application No. 63/118,754, filed on Nov. 27, 2020, the entire content of which is incorporated herein by reference.

### BACKGROUND OF THE INVENTION

All living cells in the human body are subjected to multiple modes of deformation. Mechanical factors play a critical role in the development, maintenance, degeneration, and repair of load-bearing tissues, including hard tissues (e.g., bone), and soft tissues (e.g., cartilage, tendons/ligaments). In this regard, development of reliable models to study the menchanobiology of the residing cells under physiologic and pathologic loading conditions may provide important insights into the prevention and treatment of tissue injuries and degeneration. Hydrogels, hydrophilic polymer networks, possess a vast range of mechanical properties and can be modified to biomimic the three-dimensional microenvironment of the residing cells. Furthermore, mechanical stimulation can be transmitted through the deformation of hydrogel to the encapsulated cells.

Culturing cells in a mechanically active environment can simulate the biomechanical environment in vivo, and reduce the systematic complicity relative to the use of animal models.

A number of commercial entities provide bioreactors, either in providing mechanical stimulation to cell cultures and biomaterials, including: Flexcell® (Burlington, N.C.), Bose ElectroForce® (New Castle, Del.), BISS Tissue Growth Technologies (Bangalore, India), and UStretch® (Waterloo, ON, Canada).

For instance, the Flexcell® Tension Systems are computer-regulated bioreactors that apply cyclic or static strain to cells cultured on a pneumatically deformed membrane in vitro. TA ElectroForce® BioDynamic® test instruments combine bioreactor chambers with mechanical test instruments to provide stimulation, characterization, and tissue growth solutions for engineered tissues and biomaterials within a sterile cell culture media environment. The UStretch® provides both vertical and horizontal testing in or out of a temperature-controlled media bath with a wide range of specimen attachments, including screw-driven clamps, spring-loaded clamps, and multi-point puncture grips. Though the cell culture media condition has been provided in these systems, the volume of the bathing medium is usually insufficient to sustain cells for a long period. There is difficulty in adding or removing medium, and it is difficult to optically examine the sample during dynamic compression. Additionally, the clamps used in most systems for fixing and stretching cells have limited the application in soft hydrogels. These bioreactors are only suitable either for monolayer 2D culture, or for applying mechanical loads at the tissue level. Thus, there is a need for the development of advanced research platforms to provide reliable tissue-mimetic, mechanically active, 3D microenvironments for biomedical research.

### BRIEF SUMMARY OF THE INVENTION

The present invention relates to a device and methods for inducing tensile strain in a three-dimensional (3D) cell and/or tissue culture, more specifically, in a hydrogel-magnet based cell and/or tissue culture assembly through which the stretchable hydrogel deforms in magnetic field for applying tensile strain to hydrogel-encapsulated cells and/or tissues. The subject device is user friendly; accepts soft hydrogel; allows for ease of sample loading, mechanical loading, addition and removal of growth medium, addition of bioactive agents, and allows for viewing of the samples.

The present invention further provides a device and methods for testing a fatigue property of a hydrogel-based construct, more specifically, in a hydrogel-magnet based assembly through which the stretchable hydrogel deforms in magnetic field for applying programmable tensile strain to the hydrogel. The device and methods can be used to determine the fatigue property of the hydrogel-based construct by measuring tensile force, the Young's modulus, and/or mass change and/or identifying morphological changes of the hydrogel-based construct.

The first aspect of the present invention is to provide a system for culturing cells or tissues embedded in a hydrogel-based construct or for applying mechanical stimuli to a hydrogel-based construct, wherein the hydrogel-based construct comprises a main body and at least one arm extending from the main body and carrying magnetic beads at a free end of the arm, the system comprising:
a mold for forming and shaping the hydrogel-based construct, wherein the mold has a main body concave conforming the dimension of the main body of the hydrogel-based construct, and an arm concave conforming the dimension of the arm of the hydrogel-based construct; and
a magnetic attraction force-producing device configured to cyclically apply magnetic attraction force to the magnetic bead at the free end of the arm.

In a specific embodiment, the magnetic attraction force-producing device is a rail slider comprising:
a rail,
a permanent magnet disposed on the rail, and
a platform disposed on the rail for holding the hydrogel-based construct,
wherein at least one of the permanent magnets and the platform is configured to cyclically move towards the other, thereby cyclically applying magnetic attraction force to the magnetic bead at the free end of the arm.

Preferably, the rail slider further comprises a controller configured to control an operation parameter of the rail slider.

More preferably, the operation parameter of the controller configured to control the rail slider is a minimal distance between the permanent magnet and the free end of the arm when at least one of the permanent magnets and the platform moves towards the other, a moving speed of the permanent magnet and/or the platform, or a cycle period.

In another specific embodiment, the magnetic attraction force-producing device comprises an electromagnet and a platform for holding the hydrogel-based construct, and the electromagnet is configured to be cyclically activated and deactivated, thereby cyclically applying magnetic attraction force to the magnetic bead at the free end of the arm.

Preferably, the magnetic attraction force-producing device further comprises a controller configured to control an operation parameter of the force-producing device.

More preferably, the operation parameter of the controller configured to control the force-producing device is a magnetic intensity of the electromagnet when activated, a distance between the electromagnet and the free end of the arm, or an activation cycle period of the electromagnet.

In yet another specific embodiment, the mold is formed of a polymeric material.

Preferably, the polymeric material is polydimethylsiloxane (PDMS).

Preferably, the main body concave and/or the arm concave is formed using a soft-lithography method and/or 3D printing.

In a further specific embodiment, the hydrogel-based construct is formed from a material selected from the group consisting of Gelatin Methacryloyl (GelMA), collagen, poly(ethylene glycol) diacrylate (PEGDA), methacrylated hyaluronic acid (MeHA), methacrylated chondroitin sulfate, methacrylamide chitosan (MAC), Methacrylated alginate, methacrylate and lysine functionalized dextran (Dex-MA-Ly), methacrylated gellan gum, methacrylated glycol chitosan (MeGC), Poly(ethylene oxide) (PEO), and/or poly(ethylene glycol) (PEG).

In a further specific embodiment, the system is for culturing the cells, tissues, or a combination of cells and tissues embedded in the hydrogel-based construct.

Preferably, the cell or tissue is embedded in the arm of the hydrogel-based construct.

Preferably, the cell or tissue is selected from the group consisting of a chondrocyte, a tenocyte, a mesenchymal stem cell, a stem cell, a bone marrow derived stem cell (BMSC), a meniscus progenitor cell (MPC), a tendon stem cell, a stem cell derived cell, a somatic cell, a cancer cell, a muscle cell, a nerve cell, an intestinal epithelial cell, an organoid, and a tissue explant.

The second aspect of the present application is to provide a system for applying mechanical stimuli to a hydrogel-based construct or culturing a cell or tissue embedded in a hydrogel-based construct, comprising:
a hydrogel-based construct, wherein the hydrogel-based construct comprises a main body and at least one arm extending from the main body and carrying magnetic beads at a free end of the arm; and
a magnetic attraction force-producing device configured to cyclically apply magnetic attraction force to the magnetic beads at the free end of the arm.

The third aspect of the present application is to provide a method of testing a fatigue property of a hydrogel-based construct comprising:
i) providing the hydrogel-based construct, wherein the hydrogel-based construct comprises a main body and at least one arm extending from the main body and carrying magnetic beads at a free end of the arm;
ii) providing a magnetic attraction force-producing device configured to cyclically apply magnetic attraction force to the magnetic bead at the free end of the arm;
iii) operating the magnetic attraction force-producing device, thereby cyclically applying magnetic attraction force to the magnetic bead at the free end of the arm; and
iv) determining the fatigue property of the hydrogel-based construct by measuring tensile force, the Young's modulus, and/or mass change; identifying morphological changes of the hydrogel-based construct; and/or evaluating the hydrogel degradation from quantify the released hydrogel fragments.

In a specific embodiment, step i) comprises forming the hydrogel-based construct with a mold, wherein the mold has a main body concave conforming the dimension of the main body of the hydrogel-based construct and an arm concave conforming the dimension of the arm of the hydrogel-based construct.

Preferably, step i) further comprises:
adding a first fluidic hydrogel material to the main body concave, and optionally adding an anchoring part to the main body concave, wherein the anchoring part is configured to hold the main body in position when the magnetic attraction force is applied;
solidifying the first fluidic hydrogel material;
adding the magnetic beads to the arm concave;
adding a second fluidic hydrogel material to fix the magnetic beads; and solidifying the second fluidic hydrogel material.

In another specific embodiment, the cyclically applied magnetic attraction force of the magnetic bead toward and away from the magnetic attraction force-producing object occurs at a frequency of about 0.1 Hz to about 10 Hz.

In yet another specific embodiment, the method is performed at a temperature of about 10° C. to about 50° C.

In a further specific embodiment, the cyclically applied magnetic attraction force of the magnetic beads toward and away from the magnetic attraction force-producing object occurs continuously for about 5 minutes to about 24 hours per day.

The fourth aspect of the present application is to provide a method of culturing cells or tissues embedded in a hydrogel-based construct, comprising:
i) providing the hydrogel-based construct, wherein the hydrogel-based construct comprises a main body and at least one arm extending from the main body, wherein the arm carries magnetic beads at a free end of the arm and the cell or tissue is embedded in the arm of the hydrogel-based construct;
ii) providing a magnetic attraction force-producing device configured to cyclically apply magnetic attraction force to the magnetic bead at the free end of the arm; and
iii) operating the magnetic attraction force-producing device, thereby cyclically applying magnetic attraction force to the magnetic beads at the free end of the arm.

In a specific embodiment, step i) comprises forming the hydrogel-based construct with a mold, wherein the mold has a main body concave conforming the dimension of the main body of the hydrogel-based construct, and an arm concave conforming the dimension of the arm of the hydrogel-based construct

Preferably, step i) comprises
adding a first fluidic hydrogel material to the main body concave, and optionally adding an anchoring part to the main body concave, wherein the anchoring part is configured to hold the main body in position when the magnetic attraction force is applied;
solidifying the first fluidic hydrogel material;
adding the magnetic beads to the arm concave;
adding a second fluidic hydrogel material to fix the magnetic beads;
solidifying the second fluidic hydrogel material;
adding a third fluidic hydrogel material comprising the cell; and
solidifying the third fluidic hydrogel material.

In another specific embodiment, the cyclically applied magnetic attraction force of the magnetic bead toward and away from the magnetic attraction force-producing object occurs at a frequency of about 0.1 Hz to about 10 Hz.

In yet another specific embodiment, the method is performed at a temperature of about 10° C. to about 50° C.

In a further specific embodiment, the cyclically applied magnetic attraction force of the magnetic beads toward and away from the magnetic attraction force-producing object occurs continuously for about 5 minutes to about 24 hours per day.

In a further specific embodiment, the embedded cells or tissues are selected from the group consisting of a chondrocyte, a tenocyte, a mesenchymal stem cell, a stem cell, a bone marrow derived stem cell (BMSC), a meniscus progenitor cell (MPC), a tendon stem cell, a stem cell derived cell, a somatic cell, a cancer cell, a muscle cell, a nerve cell, an intestinal epithelial cell, an organoid, and a tissue explant.

The fifth aspect of the present application is to provide a cell or tissue culturing kit, comprising
a hydrogel-based construct, wherein the hydrogel-based construct comprises a main body and at least one arm extending from the main body and carrying a magnetic bead at a free end of the arm, and the cell or tissue is embedded in the arm of the hydrogel-based construct; and
a mold for forming and shaping the hydrogel-based construct, wherein the mold has a main body concave conforming the dimension of the main body of the hydrogel-based construct, and an arm concave conforming the dimension of the arm of the hydrogel-based construct.

### BRIEF DESCRIPTION OF THE DRAWINGS

FIG. 1A-1C show a manufacture of the hydrogel construct of the 3D tensile training bioreactor. (FIG. 1A) To shape the hydrogel bioreactor, a PDMS mold was prepared by soft-lithography method, with a cuboid concave (2×0.7×0.6 cm for L×W×H) section and three connected thin concave sections (1×0.3×0.15 cm for L×W×H). (FIG. 1B) After hydrogel shaping and removal, the resulting hydrogel bioreactor has one anchoring point with a cylindrical tube (2 cm in length with a diameter of 2 mm) embedded inside in order to anchor the whole hydrogel bioreactor in the culture dish; and three arms with magnetic beads (50-100 μm in diameter, 5-10 mg/arm) fixed at the free end of each arm, which was designed for cell loading and for receiving remote tensile stimulation. (FIG. 1C) A flow chart that shows the detailed steps for manufacturing the hydrogel bioreactor, including the order to position each element and method to merge all of the elements.

FIG. 2 shows the loading station of the 3D cell tensile training bioreactor. A panorama view of the loading station displays each component. The controller allows program editing for the parameters set for controlling the speed and moving distance of the platform. The platform is used to position the cell culture plate. Magnets were fixed to provide tensile stimulation to the magnetic beads embedded hydrogel construct through the magnetic field. The controller 10 has one screen 14 for precisely displaying the moving distance of the platform, and buttons 15-19 for program editing and precisely controlling for the movement of platform 12. Under precise control, the platform 12 cyclically moves toward and away from the fixed magnet 13 at the left side of the rail slider 11 along the roller 20 using a power supply 9.

FIGS. 3A-3C show optimization and biocompatibility of the 3D cell tensile training bioreactor. (FIG. 3A) To optimize the elongation parameters of the hydrogel construct, Gelatin methacryloyl (GelMA) was used as an example hydrogel and 10-45% elongation can be achieved under tensile strain. (FIG. 3B) Live and Dead staining was used to assess the biocompatibility of the hydrogel construct, showing the cell viability after embedded in the GelMA construct. (FIG. 3C) Flow cytometry was used to test the cell viability under tensile loading. After 15 days of tensile stimulation, cells were isolated from the hydrogel constructs and stained with Propidium iodide. Cell viability is maintained under cyclic tensile stimulation when compared with the static control group.

FIGS. 4A-4B show an application of the 3D tensile training bioreactor. (FIG. 4A) Meniscus (fibrocartilage tissue in knee joint, mechanical sensitive) progenitor cells were used as an example to verify the application of the bioreactor. Extracellular matrix (ECM) secretion of cells with and without tensile stimulation was evaluated by Safranin O staining (a histology staining method to stain the cartilage ECM proteoglycan in red). (FIG. 4B) The area fraction of ECM secretion was calculated, demonstrating that ECM secretion was significantly increased under tensile stimulation, compared with the static control group. The results demonstrate the practicality application of the designed 3D tensile training bioreactor in biomedical R&D.

FIG. 5 shows how to load cyclic tensile strain on the cells in hydrogel—the design principle of the device through the following steps: a) seed cells in hydrogel (GelMA), and fix magnetic beads in the end of gel strip by UV crosslinking; b) magnetic beads stretch the cell-hydrogel strip towards the magnetic field; c) release the cell-hydrogel construct from stretching by removing the magnetic field.

FIG. 6 shows the use of a bioreactor to provide precisely controllable cyclic tensile load to hydrogel-based constructs.

FIGS. 7A-7C shows how cyclic loading accelerates hydrogel degradation. (FIG. 7A) Tissue structure of cell-hydrogel construct with or without loading at days 0, 5, 10 and 15 is stained with Picrosirius Red; Bar=100 μm. (FIG. 7B) Porosity of the cell-GelMA construct with and without tensile stimulation estimated from the Picrosirius Red staining pattern (red for thick fibers, and pink for thin fibers), and cyclic tensile load significantly enhanced GelMA hydrogel degradation (6 gel constructs/group, and 3 high magnification field of views were collected and quantified from each construct, *p<0.05). (FIG. 7C) Quantitative measurement of the pore size and distribution of the cell-GelMA constructs with and without tensile stimulation, representing GelMA degradation.

FIGS. 8A-8B show cellular readouts of cellular mechano-responsiveness and senescence. (FIG. 8A) Cell surface marker profile was assessed by flow cytometry, indicating increased cell differentiation (CD90/73/105) and mechanical sensing (CD29/49e/44) under tensile stimulation (representative data, experiment repeated >3 times). (FIG. 8B) SA-β-Gal staining results showed fewer senescent cells after 15 days of cyclic tensile loading.

FIGS. 9A-9B show bioreactor optimization and cell viability. (FIG. 9A) Optimizing GelMA concentration Young's modulus obtained by tensile testing (n=3-6, mean ±SD): 10% of GelMA with 60% degree of substitution is the most stable soft hydrogel (FIG. 9B) Human meniscus progenitor cells were isolated, and generated by multi-colony formation from pooled meniscus cells (n=9, 3 batches), and the colony-formation-unit (CFU) seen by Crystal Violet staining.

### DETAILED DISCLOSURE OF THE INVENTION

### Definitions

Ranges provided herein are understood to be shorthand for all of the values within the range. For example, a range of 1 to 20 is understood to include any number, combination of numbers, or sub-range from the group consisting 1, 2, 3, 4, 5, 6, 7, 8, 9, 10, 11, 12, 13, 14, 15, 16, 17, 18, 19 and 20, as well as all intervening decimal values between the aforementioned integers such as, for example, 1.1, 1.2, 1.3, 1.4, 1.5, 1.6, 1.7, 1.8, and 1.9. With respect to sub-ranges, “nested sub-ranges” that extend from either end point of the range are specifically contemplated. For example, a nested sub-range of an exemplary range of 1 to 50 may comprise 1 to 10, 1 to 20, 1 to 30, and 1 to 40 in one direction, or 50 to 40, 50 to 30, 50 to 20, and 50 to 10 in the other direction.

As used herein a “reduction” means a negative alteration, and an “increase” means a positive alteration, wherein the negative or positive alteration is at least 0.001%, 0.01%, 0.1%, 0.5%, 1%, 5%, 10%, 15%, 20%, 25%, 30%, 35%, 40%, 45%, 50%, 55%, 60%, 65%, 70%, 75%, 80%, 85%, 90%, 95% or 100%.

The transitional term “comprising,” which is synonymous with “including,” or “containing,” is inclusive or open-ended and does not exclude additional, unrecited elements or method steps. By contrast, the transitional phrase “consisting of” excludes any element, step, or ingredient not specified in the claim. The transitional phrase “consisting essentially of” limits the scope of a claim to the specified materials or steps “and those that do not materially affect the basic and novel characteristic(s)” of the claimed invention. Use of the term “comprising” contemplates other embodiments that “consist” or “consist essentially of” the recited component(s).

Unless specifically stated or obvious from context, as used herein, the term “or” is understood to be inclusive. Unless specifically stated or obvious from context, as used herein, the terms “a,” “and” and “the” are understood to be singular or plural.

Unless specifically stated or obvious from context, as used herein, the term “about” is understood as within a range of normal tolerance in the art, for example within 2 standard deviations of the mean. About can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein are modified by the term about.

The present invention provides a user friendly system and methods to apply physical forces to hydrogels, cells and/or tissues. The resulting cells and/or tissue can be used to analyze biochemical and biomechanical responses in cells encapsulated in hydrogels in a defined configuration. The subject cell culture tensile device and methods can be used to apply tensile strain to cells or tissue explants either encapsulated in the hydrogel directly or seeded on the hydrogel-embedded scaffold so that allows analysis of the response of cells to an applied tensile strain and interfacial stress. In the presence or absence of various chemical mediators, the novel mechanical loading system is designed to allow for continuous assessment of multiple outcome measures, such as cell viability, reproduction and metabolic activity, cell morphology, extracellular matrix activity, and cell signaling. The device has the ability to culture cells and/or tissues in a hydrogel-based 3D environment, and apply the mechanically load in a contact-free way. The subject device and methods have broad applications in many fields including stem cells, genomics, tissue engineering, pharmacology, regenerative medicine, and biotechnology. The subject device may be used as a tool used for analyzing differential cell mechanosensitive responses of normal and pathologic cells in medicine.

### Tensile Device and Methods of Use

In certain embodiments of the present invention provide a hydrogel-based tensile device which can include: (i) a rail slider with a platform and controller; (ii) a magnet fixed on the rail slider; (iii) a mold for shaping and setting-up a hydrogel construct with embedded magnetic beads and said hydrogel; and/or (iiii) a cell/tissue culture dish positioned on the platform, wherein the shaped hydrogel construct containing cells and/or tissues is cultured in a growth medium. Incorporating various hydrogels, the tensile device provides cyclic tensile strain to the hydrogel within a controllable distance and loading range. In certain embodiments, the tensile device provides cyclic tensile strain to hydrogel-encapsulated cells within a controllable distance and loading range.

In certain embodiments, the present invention provides a tensile system comprising hydrogel construct with embedded magnetic beads, wherein the hydrogel is anchored to an anchoring point. The anchoring point can be a cell culture dish or any other type of container. The tensile system can further comprise a magnet field-fixed motorized rail slider (MRS) controller, a rail slider that can support a hydrogel construct in, optionally, a cell and/or tissue culture container, and/or a mold for shaping the hydrogel. The hydrogel can be elongated under the magnetic field provided by the magnet-magnetic beads interaction. With cells and/or tissues loaded in the hydrogel, the elongation of arms can deform the loaded cells and/or tissues.

According to an embodiment, the present invention provides a hydrogel-based tensile system comprising magnet-fixed rail slider and corresponding controller. The rail slider can alternatively be any device that permits a repeated interaction between a fixed magnet, including an electromagnet, and magnetic particles embedded in a hydrogel, including but not limited to, a bearing slider, a roller slider, a turntable, a rotating tray, a lazy Susan, a conveyor (e.g., belt conveyor, chain conveyor), a wheel, and/or a roller. In preferred embodiments, a sliding mechanism facilitates the transfer of the driving movement in one direction and the return in the opposite direction. In certain embodiments, the rail slider or alternative device can support a platform in or on which the hydrogel and/or cell/tissue culture ultimately resides.

In certain embodiments, the movement of the rail slider or alternative device can be modulated by a controller and a motor. The controller can allow for the editing of parameters for the movement of the hydrogel-based construct, such as controlling the movement of a platform on which a petri dish containing a hydrogel-based construct resides along a rail slider. The controller can have at least one screen 14 for precisely displaying the moving distance of the platform, and buttons 15-19 for program editing and precisely controlling for the movement of platform 12. Alternatively, the controller can be modulated remotely, i.e., by a remote control, smartphone application, and other related technologies. Under precise control, the platform 12 cyclically can move toward and away from the fixed at least one magnet 13 along the roller 20. In this way, the fixed magnet 13 provides an attraction force to magnetic beads embedded construct 4, allowing the application of the cyclic tensile strain to hydrogel-based bioreactor 4 or hydrogel-based construct with controlled speed and moving distance. Alternatively, the rail slider can be operated by manually, without the use of a controller and/or motor.

In certain embodiments, at least 1, 2, 3, 4, 5, 6, 7, 8, 9, 10, or more magnets can be fixed at the rail slider to provide a magnetic field. The magnet can be a conventional (permanent) magnet or an electromagnet. The magnets can interact with magnetic beads embedded in at least one hydrogel, resulting in the cyclic elongation of the at least one hydrogel that can be anchored to at least 1, 2, 3, 4, 5, 6, 7, 8, 9, 10, or more anchoring points. In certain embodiments, the magnetic induction is in the range of about 0.1 tesla (T) to about 10 T, about 0.2 T to about 1.5 T, about 0.29 T to about 0.9 T, about 0.3 T, or about 0.29 T. In other embodiments, the provided level of magnetic induction can apply a determined tensile strain to a hydrogel. In certain embodiments, the tensile strain can be about 0.005 MPa to about 10 MPa or about 0.01 MPa to about 20 MPa. Furthermore, the tensile strain can be about 0.1 MPa to about 500 MPa, about 10 MPa to about 350 MPa, or about 100 MPa to about 250 MPa. Alternatively, the tensile strain can be determined through the deformation of the hydrogel, cells, and/or tissues. The percent deformation of the hydrogel, cells, and/or tissues can be 0.01% to about 20% deformation, about 1% to about 10% deformation, or about 2% to about 7.5% deformation. In certain embodiments, a magnetic field can be provided by the fixed neodymium magnets (˜0.29 T) to attract magnetic beads embedded in the hydrogel construct, and to apply cyclic tensile stimulation for the construct. To precisely control the magnetic force-driven hydrogel elongation, the volume of the magnetic beads can be adjusted, the distances between the anchoring point and the magnet, and the magnetic field. In certain embodiments, about 5 mg to about 10 mg of magnetic beads, preferably with a diameter of about 50 μm to about 100 μm, can be added to an arm of the hydrogel.

The tensile system can further comprise cells and/or tissues encapsulated in hydrogel arms. Some examples of the loaded cells and/or tissues include, but are not limited to one or multiple cell types of chondrocyte, tenocytes, mesenchymal stem cells, stem cells, bone marrow derived stem cells (BMSC), meniscus progenitor cells (MPC), tendon stem cells, a stem cell derived cell, a somatic cell, a cancer cell, a muscle cell, a nerve cell, an intestinal epithelial cell, organoids and many other mechanical sensitive and responsive cells and tissue explants. Incorporating loaded cells, the free-contact tensile system can be used to study cell metabolism, cell differentiation, function, and cell-secretome.

The material of the mold used to shape the hydrogel using can include, but is not limited to, silicon, glass, ceramic, elastomers including, but not limited to, acrylic, thermoplastic polymers including poly(methyl methacrylate) (PMMA), poly(dimethylsiloxane) (PDMS), polystyrene, polyurethane, thermoset polyester, polycarbonate, cyclo-oelfin polymer (COP), poly(methyl glutarimide) (PGM1), phenol formaldehyde resin, epoxy-based polymers, polyethylene teraphthalate (PET), and other polymeric materials.

According to one aspect, the present invention provides a system comprising a mold to shape a hydrogel; the resulting hydrogel can possess at least 1, 2, 3, 4, 5, 6, 7, 8, 9, 10, or more anchoring points. The anchoring points can be to the cell and/or tissue culture dish or any other type of container. The hydrogel, as determined by the mold, can also possess at least 1, 2, 3, 4, 5, 6, 7, 8, 9, 10, or more arms. In certain embodiments, cells and/or tissues can be encapsulated in the arm(s), extending from a base of the hydrogel. Cells and/or tissues can be cultured in the hydrogel, particularly in the arms extending from the base of the hydrogel and metabolism exchange can occur with growth medium in a culture dish.

The hydrogel can be cured within the aforementioned mold, or can be cured in bulk and pieces of the bulk article can be cut from the mold, as desired, for use in culture. The polymerization can be carried out by any free-radical initiation system, including any thermal, redox, or photochemical system. In preferred embodiments, the hydrogel is photo-crosslinkable Gelatin methacrylate.

The cell culture dish or any other types of cell culture container can include, but is not limited to, flasks, beakers, and tubes. The cell culture container can be silicon, glass, ceramic, elastomers including, but not limited to, acrylic, thermoplastic polymers including poly(methyl methacrylate) (PMMA), poly(dimethylsiloxane) (PDMS), polystyrene, polyurethane, thermoset polyester, polycarbonate, cyclo-oelfin polymer (COP), poly(methyl glutarimide) (PGM1), phenol formaldehyde resin, epoxy-based polymers, polyethylene teraphthalate (PET), and other polymeric materials. In particular, the device can be configured to contain at least 1, 2, 3, 4, 5, 6, 7, 8, 9, 10, or more 10-cm cell culture Petri dishes or other cell culture containers made of glass or polystyrene.

Embodiments according to this aspect can include one or more of the following features. The hydrogel-based tensile system can further comprise at least one additional scaffold embedded in the at least one hydrogel stretchable arm. Incorporating at least one cell-seeded scaffold, the hydrogel-based tensile system can further be used to study the tensile strain on cells and stress at the interface.

In certain embodiments, the hydrogel constructs can be subjected to a series of cyclic tensile loading. Cyclic tensile loading can be applied for at least about 1 min, about 2 mins, about 3 mins, about 5 mins, about 10 mins, about 15 mins, about 30 mins, about 45 mins, about 1 h, about 2 h, about 3 h, about 4 h, about 8 h, about 12 h, about 16 h, about 20 h, about 23 h or up to 24 h per day at a frequency of at least about 0.01 Hz, about 0.1, about 0.25 Hz, about 0.5 Hz, about 1 Hz, about 1.5 Hz, about 2 Hz, about 2.5 Hz, about 5 Hz, about 10 Hz, about 25 Hz, about 50 Hz or a greater at temperature of about 0° C. to about 100° C., about 5° C. to about 75° C., about 10° C. to about 50° C., 15° C. to about 30° C., about 18° C. to about 22° C., at room temperature. Cyclic tensile loading can be applied for range of about 1 min to about 24 h, about 2 mins to about 20 h, about 10 mins to about 12 h, about 30 mins to about 8 hours, or about 1 h to about 3 h, per day at a frequency in the range of about 0.01 Hz to about 50 Hz, about 0.1 to about 25 Hz, about 0.25 Hz to about 10 Hz, about 0.5 Hz to about 5 Hz, or about 0.5 Hz to about 2 Hz. In certain embodiments, the cyclic tensile loading can take place within a single day, about 2 days, about 3 days, about 4 days, about 5 days, about 6 days, about 7 days, about 10 days, about 14 days, about 15 days, about 21 days, about 28 days, about 35 days, about 42 days, about 49 days, about 56 days, about 63 days, about 70 days, about 77 days, about 84 days, about 91 days, about 98 days, about 105 days, about 110 days, about 112 days, about 119 days, about 126 days, about 133 days, about 140 days, about 147 days, about 154 days or more. In certain embodiments, the cyclic tensile loading can take place within a range of about 1 day to about 6 months, 2 days to about 5 months, 3 days to about 4 months, 4 days to about 3 months, 5 days to about 2 months, or about 6 days to about 1 month.

In certain embodiments, all tissue/cell samples (both static and loading) can be maintained in chondrogenic condition medium in incubator (37° C., 5% CO₂), and can be collected for analysis at day 0, 5, 10, and 15; alternatively, the samples can be collected twice daily, daily, every other day, every third day, or every four days. Cell viability of the encapsulated cells and/or tissues with or without tensile treatment can be assessed through Live and Dead Staining and/or Flow Cytometry by known methods in the art. Cell differentiation and senescence can be assessed by Flow Cytometry and SA-β-Gal staining, respectively. To isolate cells for staining or flow cytometry the cells can be dissociated from a hydrogel with enzymes and/or reagents. In certain embodiments, collagenase can release cells from a gelatin methacryloyl (GelMA) hydrogel after culturing, including collagenase are Type I, II, and IV. In certain embodiments, hyaluronidase can release cells from a hyaluronic acid methacrylate (HAMA) hydrogel after culturing. In certain embodiments, a sodium citrate solution (preferably with a concentration of 55 mM at a pH of 6.8) can release cells from alginate-based hydrogel after long-term culture.

### Hydrogels

Hydrogels, which are hydrophilic polymer networks, possess a vast range of mechanical properties, and have been widely used in biomedical applications. In certain embodiments, photo-crosslinkable hydrogels such as GelMA hydrogels, HAMA, alginate, and poly(ethylene glycol)-diacrylate (PEGDA) hydrogels can be used as extracellular matrix (ECM)-mimetic supportive soft scaffolds for cellular growth and tissue formation, or as bio-inks for 3D-printing and tissue engineering, owning to their advanced cell compatibility, low immunoreactivity, and tunable physical characteristics. The hydrogel can include natural and/or synthetic polymer macromers that can be cross-linked. The number or percentage of cross-links linking the macromers can be varied to control the mechanical properties, chemical properties, swelling ratios, and degradation profiles of the hydrogels. Degradation of the cross-links in vivo allows the hydrogel to more readily biodegrade and be used for in vivo applications. Additionally, the hydrogel can be used as a substrate for the incorporation and/or attachment of various agents, tissues, and/or cells. The hydrogel can be injectable and/or implantable, and can be in the form of a membrane, sponge, gel, solid scaffold, spun fiber, woven or unwoven mesh, nanoparticle, microparticle, or any other desirable configuration. The hydrogel can be a composition of one or more compounds classified as polypeptides, polysaccharides, and/or synthetic. Examples of compounds further include acrylamide, hydroxyethyl methacrylate (HEMA), agarose, methylcellulose, hyaluronan, GelMA, cyclodextrin, and/or alginate. The cell-hydrogel construct can a basic in vitro 3D tissue culture unit that can be further modified with tissue-specific bioactivity by binding bioactive motifs to the hydrogel or by adding bioactive molecules to the tissue culture medium. Furthermore, mechanical stimulation can be transmitted through the deformation of hydrogel to the encapsulated cells, and this mild mechanical load is more like that seen in native tissues; therefore, hydrogels can also be used as vehicles and scaffolds to protect and deliver cells in cell-based therapies. Taking the mechanically active environment into consideration, the cell compatible soft hydrogels, such as GelMA, can be used because they are both mechanically supportive and biodegradable, with the ability to assist tissue growth at a matched rhythm of biodegradation. In certain embodiments, the hydrogel itself can undergo mechanical stimulation through the deformation of the hydrogel without embedding cells of tissues. In preferred embodiments, GelMA, collagen, and PEGDA, HAMA, methacrylated chondroitin sulfate, methacrylamide chitosan (MAC), methacrylated alginate, methacrylate and lysine functionalized dextran (Dex-MA-Ly), methacrylated gellan gum, methacrylated glycol chitosan (MeGC), poly(ethylene oxide) (PEO), and/or poly(ethylene glycol) (PEG) can be used to create a hydrogel.

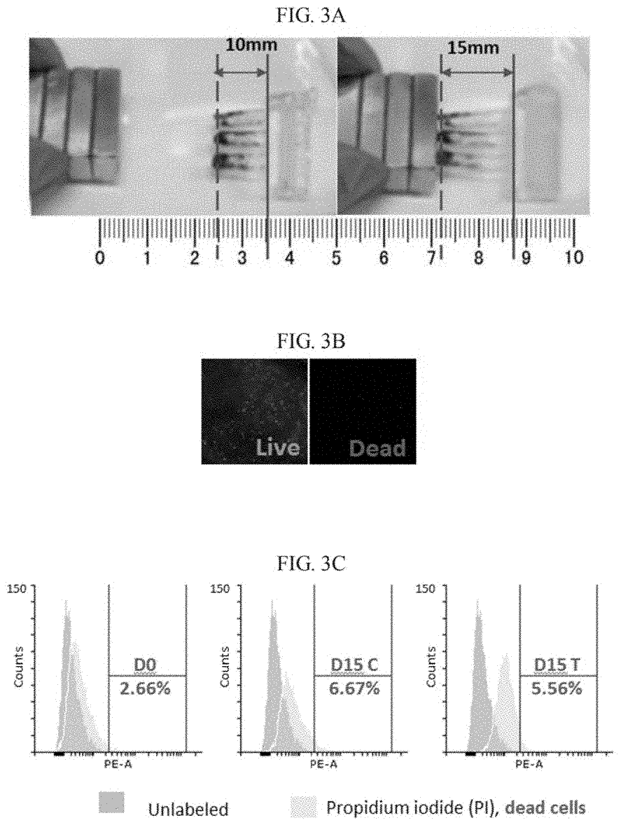

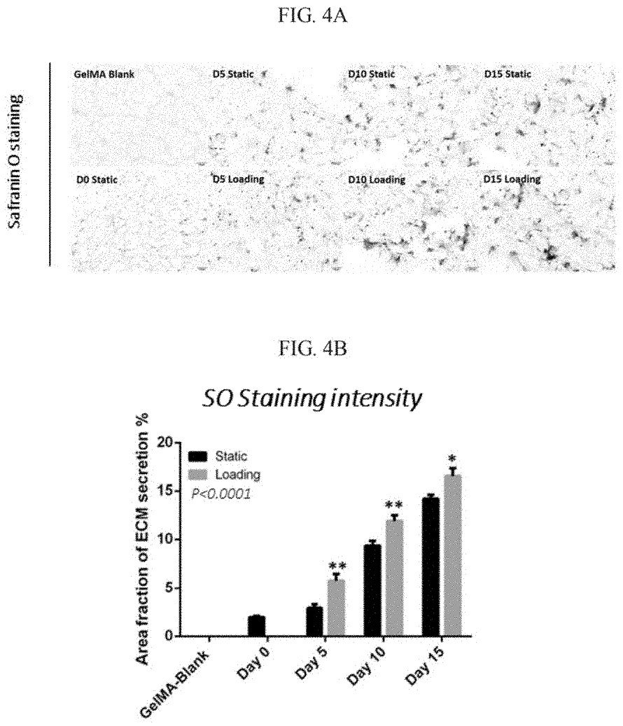

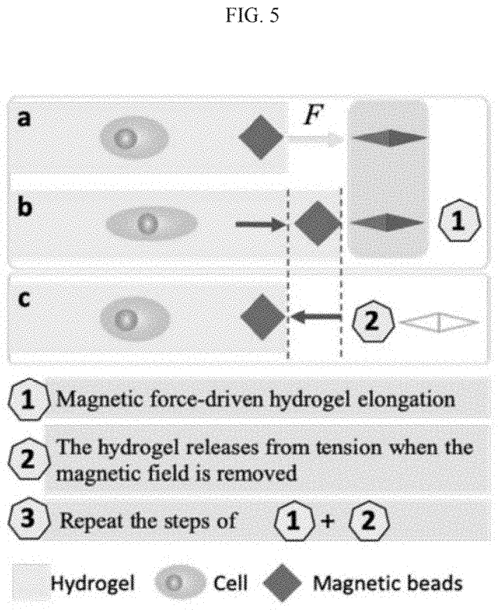

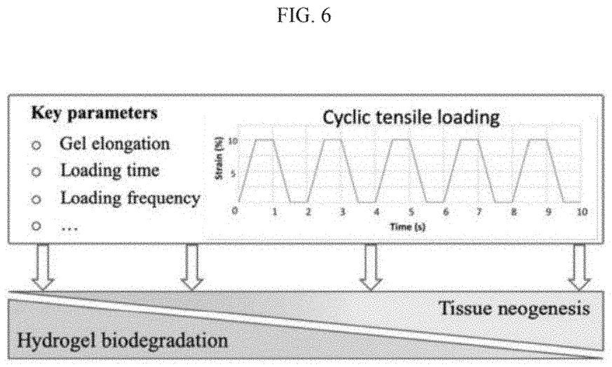

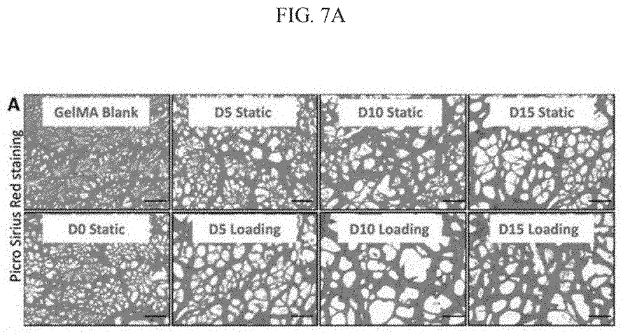

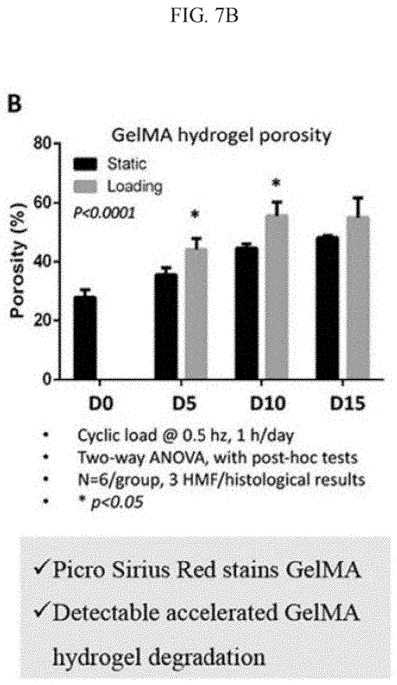

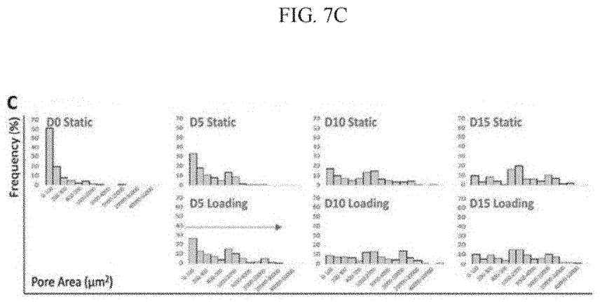

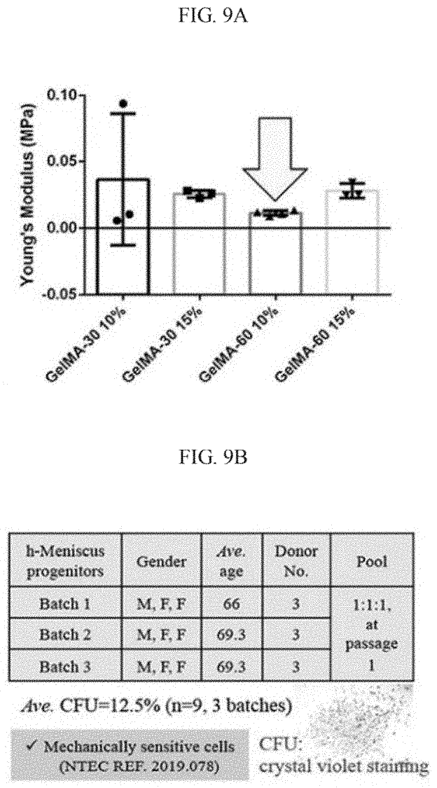

In certain embodiments, there are several key, modifiable parameters of the hydrogel and methods of mechanical loading, such as the composition and concentration (e.g., GelMA-60, 10%, FIG. 9A), cell seeding density (e.g., 5×10⁶ cells/ml, FIGS. 4A-4B), the effective range of gel elongation (e.g., 10%-45% of deformation, FIG. 3A), and the cyclic loading time (1 hour/day; 0, 5, 10, 15 days; FIGS. 7A-7C, FIGS. 4A-4B, FIGS. 8A-8B) and mechanical frequency (0.5-1 Hz; FIGS. 7A-7C, FIGS. 4A-4B, FIGS. 8A-8B). In certain embodiments, the hydrogel, can be prepared with various degrees of substitution and concentration, such as, for example GelMA-30/60/90; 5%, 7.5%, 10%, 12.5%, 15%. In certain embodiments, if the hydrogel is a photo-crosslinkable hydrogel, a photo-initiator, such as, for example lithium acylphosphonate, can be added to the hydrogel. The photo-initiator can be dissolved in PBS at 0.25% w/v and incubated in a 60° C. water bath for 30 minutes, and filtered through a 0.22-μm filter.

### Cell-encapsulation and Establishment of Cell-Hydrogel Construct

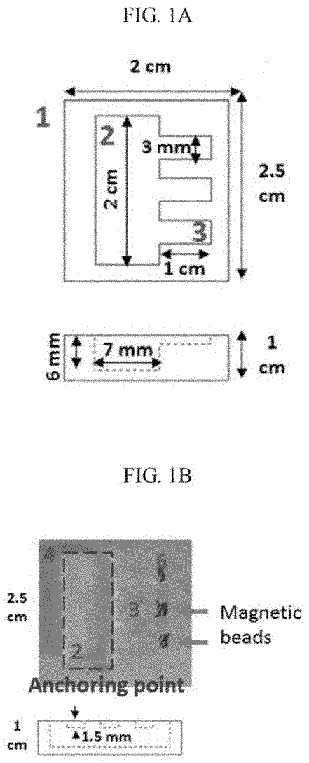

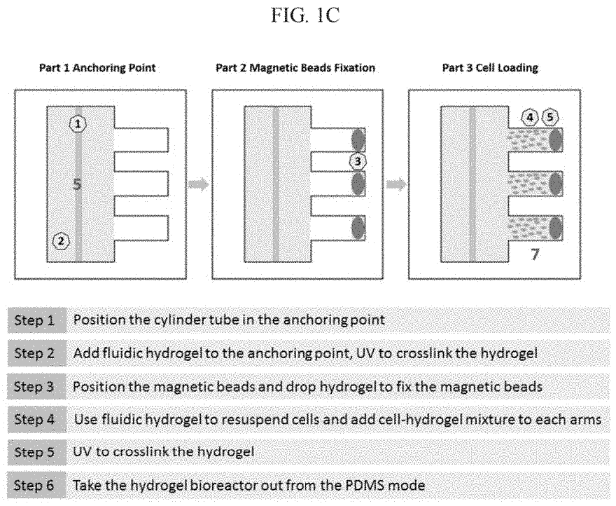

In certain embodiments, the cell-hydrogel 3D cultures can be set up in the designed mold (FIGS. 1A-1C). In brief, the cells can be detached from growth flasks using, for example, incubation of Trypsin (0.25% EDTA), and resuspended in a hydrogel solution, such as, for example, a GelMA/LAP solution, at a concentration of cells of about 0.1×10⁶ cells/ml to about 25×10⁶ cells/ml, 1×10⁶ cells/ml to about 10×10⁶ cells/ml, or about 3×10⁶cells/ml to about 6×10⁶ cells/ml and, optionally, photo-crosslinked with 365-nm UV light for 25-30 seconds in the mold made from soft lithography. The produced hydrogel constructs can have one, two, three, four, five, or more anchoring points with the optional dimensions of 2 cm×7 mm×6 mm for L×W×H and at least one, two, three, four, five, six, or more arms with the optional dimensions of about 10 mm×3 mm×1.5 mm (L×W×H); 5-10 mg magnetic beads with diameters of about 50 μm to about 500 μm, about 50 μm to about 300 μm, or about 50 μm to about 100 μm can be placed at the end of each arm. In certain embodiments, if the hydrogel construct contains cells and/or tissues, the hydrogel construct can be cultured in general cell culture medium, such as, for example, Dulbecco's modified Eagle's medium [DMEM] with 10% fetal bovine serum and/or chondrogenic condition medium (serum-free, high glucose DMEM with 1% antibiotics-antimycotic, insulin-transferrin-selenium [ITS], 40 μg/ml proline, 50 μg/ml ascorbic acid, 0.1 μM dexamethasone, and 10 ng/mL transforming growth factor beta 3 [TGF-β3]) to induce the ECM deposition.

### Cyclically Applied Forces to Hydrogel Construct

In certain embodiments, the subject methods and systems can be used to determine hydrogel fatigue and deformation in addition to using the methods and systems to simulate in vivo forces on cells and/or tissues. The hydrogels can be loaded into the system of the subject invention, and the hydrogel elongation and deformation before and after relaxation from the cyclic load, can be recorded using a high-speed camera. In certain embodiments, part of the hydrogel fatigue can be reflected by the gradual changes in the Young's modulus, the altered gel-elongation, and relaxation sites.

In some embodiments, tension testing of the hydrogels before and after cyclic loading will be performed using a rheometer modified with parallel mechanical clamp. The tensile mechanical test can be conducted at an extension speed of 1 mm/s to record the tensile force, and the Young's modulus can be evaluated and calculated. Other hydrogel degradation related assays such as mass loss (by wet weight) and morphological changes (by scanning electron microscope, SEM) can be conducted. Hydrogels can be modified by introducing natural-derived compositions (e.g., hyaluronic acid (HA), chondroitin sulfate (CS), and tissue-derived ECM into the hydrogels). In some embodiments, fibrin or other soluble tendon-derived decellularized ECM can be added to a hydrogel.

In certain embodiments, the degradation rate of hydrogel constructs (with or without cells/tissues; with or without cyclic tensile load) can be examined with the use of a florescent tagged hydrogel. The construct can be observed via fluorescence imaging, and the basing medium can be collected daily and evaluated with a microplate reader for fluorescence.

In certain embodiments Histological assays will be used to test hydrogel degradation with or without cyclic tensile stimulation. Hydrogel constructs (with or without cells/tissues) can be fixed, and embedded in optimal cutting temperature compound and cryosectioned at a thickness of about 10 μm.

### MATERIALS AND METHODS

### Isolation and Identification of Human Meniscus Progenitor Cells

Human meniscus specimens were obtained from patients undergoing total knee arthroplasty surgeries. Human meniscus cells were isolated through collagenase I incubation (e.g., n=9), which were further expanded and seeded at low density (10 cells/cm²) to form colonies. The putative cells after colony selection were designated as meniscus progenitor cells, and passage 1-4 were used for all experiments. Crystal Violet Staining was used to count the number of formed colonies, and Flow Cytometry was used to identify the meniscus progenitor cells through MSC surface markers.

### Mechanical Stimulation and Cell Mechanobiological Response

Cyclic tensile loading was applied for 1 h/day at a frequency of 0.5 Hz at room temperature. All samples (both static and loading) were maintained in chondrogenic condition medium in incubator (37° C., 5% CO₂), and were collected for analysis at days 0, 5, 10, and 15. Cell viability of the encapsulated cells with/ without tensile treatment was assessed through both Live and Dead Staining and Flow Cytometry. Cell differentiation and senescence were assessed by Flow Cytometry and SA-β-Gal staining, respectively.

### Analyzing Hydrogel Degradation and Tissue-Specific ECM Re-Establishment

Hydrogel degradation and/or new tissue ECM generation can be evaluated by histological analysis, such as picrosirius Red (PSR) to present the structure of the hydrogel at different time points, and a histology staining method to stain the cartilage ECM proteoglycan using, for example, red Safranin O staining to quantify the pericellular ECM deposition over time. Additionally, fluorescent tagged hydrogel and molecular tracing tools such as fluorescence non-canonical amino acid tagging (FUNCAT) staining can be used to evaluate hydrogel degradation and new tissue ECM generation. Fluorescent tags can include, for example, Alexa Fluor® 488; phycoerythrin (PE); PerCP-Cy5.5; PE-Cy7; (Allophycocyanin) APC; rhodamine and its derivatives (e.g., TRITC, TAMRA, Rhodamine B, Rhodamine 6G, RhBITC, Dihydrorhodamine, Sulforhodamine, Tetramethylrhodamine-6-maleimide, Tetramethylrhodamine-5-maleimide); fluorescein and its derivatives (e.g., FITC, Fluorescein-5-maleimide, 5-IAF, 6-TET, 6-FAM); coumarin and its derivatives (e.g., 7-Hydroxy-4-methylcoumarin, 3-Cyano-7-hydroxycoumarin, AMC); and/or green fluorescent protein (GFP).

### Cell Types, Preparation, and Characterization

We have isolated and stocked three batches of tissue-specific progenitor cells from human meniscus (FIG. 9B) in liquid nitrogen tank (collected and isolated from arthroplasty following our established standard operating procedures with IRB approval), and we have also isolated and cultured the cells from other mechanically sensitive tissues such as tendon and bone, and prepare bone marrow-derived mesenchymal stem cells (MSCs) to study the application of the bioreactor on 3D cultured stem cells. The progenitor cells were selected by CFU ability based on their self-renewal ability. For meniscus progenitors, cells from the age and gender matched donors were pooled into three batches (detailed information is listed in FIG. 9B). After passage 1, isolated cells from each batch were seeded at a low density on T175 cell culture flasks or a 10-cm cell culture dish (10 cells/cm²) to form colonies. Fourteen days after initial seeding, the colonies that formed on 10-cm culture dishes were stained with 1% crystal violet solution. The number of colonies formed was counted as the CFU. The putative cells after colony selection were designated as meniscus progenitor cells, and passages 2-6 were used for all experiments and will be used in further studies. For progenitor cells from tendon and other tissues, a similar pooling strategy with multi-colony forming selection will be performed, and the cells will be characterized by CFU and flow cytometry.

All patents, patent applications, provisional applications, and publications referred to or cited herein are incorporated by reference in their entirety, including all figures and tables, to the extent they are not inconsistent with the explicit teachings of this specification.

Following are examples that illustrate procedures for practicing the invention. These examples should not be construed as limiting. All percentages are by weight and all solvent mixture proportions are by volume unless otherwise noted.

### EXAMPLE 1—MANUFACTURE OF THE HYDROGEL CONSTRUCT OF 3D TENSILE TRAINING BIOREACTOR

As shown in FIG. 1A, one embodiment of the cell culture tensile device includes a PDMS mode 1, a 2.5 cm×2 cm×1 cm cube with grooves made by soft-lithography method. The grooves formed a sunken space for shaping the hydrogel, which comprises two parts: one anchoring point 2 with the dimension of 2 cm×0.7 cm×0.6 cm, and three cell loading arms 3 with dimension of 10 mm×3 mm×1.5 mm in each arm. The shaped elastic hydrogel bioreactor 4 with the designed PDMS mode 1 was shown in FIG. 1B. The hydrogel biomaterial used for constructing a 3D cell tensile training bioreactor 4 should have adjustable stiffness, controllable solidification, and allow cells to proliferate and spread. The examples of the suitable hydrogel include, but are not limited to Gelatin Methacryloyl (GelMA), collagen, and poly(ethylene glycol) diacrylate (PEGDA). In the anchoring point 2 of the shaped hydrogel-based construct exists a cylinder tube 5 (2 mm in diameter and 2 cm in length) for anchoring, and 5-10 mg magnetic beads 6 with diameter 50-100 μm are positioned at the unconnected end of cell loading arms 3. To set up the 3D cell tensile training bioreactor, cylinder tube 5 is firstly positioned in the sunken space for anchoring point 2 in the PDMS mode 1, and magnetic beads 6 are positioned at the unconnected end of the sunken space in each cell loading arm 3. Fluidic hydrogel is then used to fill the sunken space of anchoring point 2 in the PDMS mode 1 and to fix the magnetic beads 6 in place. The fluidic hydrogel-cell mixture 7 fills the remaining sunken space in cell loading arms 3. After solidifying, the shaped 3D cell tensile training bioreactor 4 is taken out from the PDMS mode 1.

### EXAMPLE 2—LOADING STATION OF 3D CELL TENSILE TRAINING BIOREACTOR

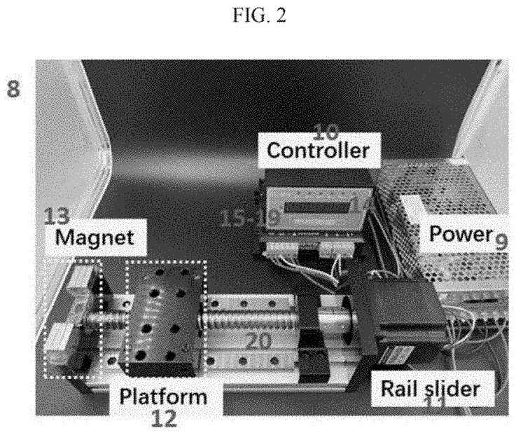

Referring to FIG. 2, the cell culture tensile loading station 8 comprises a power source 9, a controller 10, a rail slider 11, a platform 12, and at least one magnet 13 fixed at the left side of the rail slider 11. The controller 10 allows program editing for the parameters set for cyclic movement of the platform 11, and has one screen 14 for precisely displaying the moving distance of the platform, and five buttons 15-19 for program editing and precisely controlling for the movement of platform 12. Under the precisely control, the platform 12 cyclically moves toward/away from the fixed at least one magnet 13 along the roller 20. In this way, the fixed magnet 13 provides an attraction force to magnetic beads embedded construct 4, allowing the application of the cyclic tensile strain to hydrogel-based bioreactor 4 with controlled speed and moving distance. The cell culture dish containing 3D cell tensile bioreactor 4 can be positioned on the platform 12 for free-contact cyclic tensile strain.

As such, the present invention provides a method to make free-contact cell tensile device which can include the following three general components: one or more PDMS molds 1 for shaping the hydrogel, one or more magnetic beads-embedded hydrogel bioreactors 4 for cell culturing and strain receiving, and one cell tensile loading station 8 to apply tensile strain to the hydrogel-based bioreactor 4. By further incorporating cells encapsulated in the loading arms 3, the resulting assembled device can provide (a) a 3D cell culture environment, (b) a free-contact way for remotely applying tensile strain, and (c) cyclic tensile strain for cell culture. In addition, according to some embodiments, the 3D tensile device can be used to study the interfacial stress of the scaffold embedded in the hydrogel-based bioreactor 4 loading arms 3.

### EXAMPLE 3—OPTIMIZATION, BIOCOMPATIBILITY, AND APPLICABILITY OF THE 3D CELL TENSILE TRAINING BIOREACTOR

Meniscus tears are frequently encountered in clinical practice, and while partial or complete meniscectomy is a common treatment option, general meniscus loss is a risk factor for the development of osteoarthritis. Efforts are made to achieve seamless healing of meniscus tears, mainly involving the usage of biocompatible hydrogel and tissue specific stem/progenitor cells. As a load-bearing tissue, the dynamics of meniscus repair in vivo are governed by both of biological and biomechanical cues. To mimic authentic load-bearing meniscus, dynamic mechanical stimulation is indispensable, which can recapitulate the mechanical microenvironment of meniscus, regulate the release profile of the preselected growth factors, and promote the meniscus healing process.

Mechanical sensitive tissue progenitor cells, the human meniscus stem/progenitor cells (hMeSPCs), were isolated, expanded and characterized in vitro, and encapsulated in Gelatin Methacryloyl (GelMA) hydrogel. Referring to FIGS. 3A-4B, GelMA and meniscus progenitor cells were used as examples to optimize the elongation parameters of the hydrogel construct and assess the biocompatibility of the 3D tensile training bioreactor. As shown in FIG. 3A, GelMA-based construct can be elongated up to 45% under the magnetic field provided by the magnets 13 in loading station 8. FIGS. 3B-3C demonstrated the great biocompatibility of the hydrogel construct through Live and Dead staining and flow cytometry. Cell viability maintained under cyclic tensile stimulation, compared with the static control group. Furthermore, to verify the application of the 3D tensile training bioreactor, ECM secretion and deposition were assessed through Safranin O staining, and the area fraction was calculated, which were shown in FIGS. 4A and 4B, respectively. The results demonstrated that ECM secretion was significantly increased under tensile stimulation, compared with the static control group.

To mimic the mechanical environment of meniscus healing in vitro, Controlled tensile strain was applied to the hydrogel-encapsulated cells through the homemade 3D bioreactor, which enabling the 3D-cell culture of hMeSPCs for more than 15 days with 94% of the cell survival rate. We found that hMeSPCs displayed mesenchymal stem cell characteristics after isolation, and the controlled tensile strain loading enhanced the differentiation of GelMA encapsulated hMeSPCs, compared with the static group. The re-establishment of the extracellular matrix (ECM) under tensile strain loading was increased. Furthermore, less cell senescence was observed after tensile treatment, compared with the static control group. These findings show that mechanical loading assists meniscus derived progenitor cell differentiation, inhibiting cell senescence, and it is a promising advanced platform for deciphering the biomechanics in both cell and tissue levels.

### EXAMPLE 4—DECIPHERING HOW CYCLIC TENSILE LOAD REGULATES MENISCUS PROGENITOR CELL BEHAVIOR

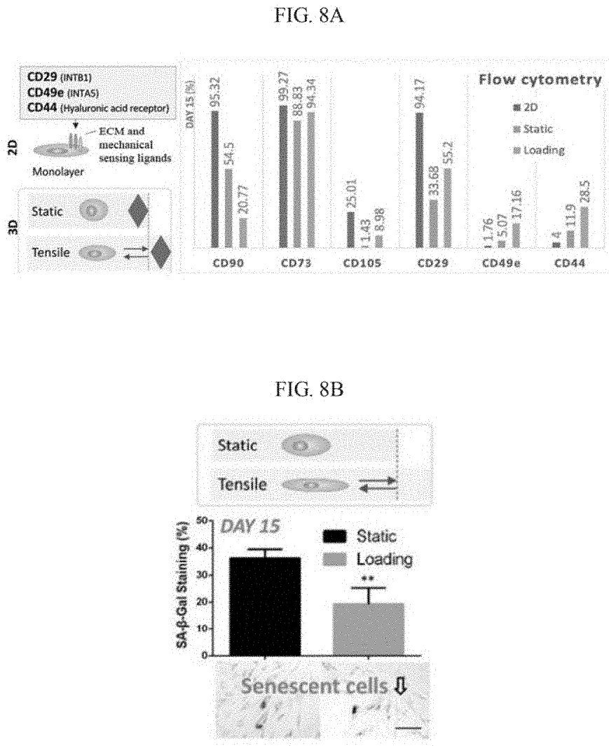

Using the subject methods and systems, we can perform unique experiments and have collected some interesting results to decipher how the cyclic tensile load regulates stem/progenitor cells. For example, we have established the methodology and protocol to release and collect the cells from the GelMA hydrogel cultures after 15 days of cyclic tensile loading (i.e., with collagenase, the enzyme to digest GelMA hydrogel). The released cells can be immediately analyzed by flow cytometry, and we have found that the 3D cyclic tensile loading changes the encapsulated cell surface marker profile, in terms of stem cell-related cell surface markers (e.g., CD90, CD73 and CD105), the ECM receptor (CD44, hyaluronic acid receptor), and mechanical sensor ligands integrin beta 1 (ITGB1, also known as CD29) and integrin alpha 5 (ITGAS, also known as CD49e) (FIG. 8A), and it is much more informative than the results from the 2D monolayer cultured control group. In addition, we can examine the cell senescence of cells released from the hydrogel and have found fewer senescent cells after cyclic tensile loading (FIG. 8B).

It should be understood that the examples and embodiments described herein are for illustrative purposes only and that various modifications or changes in light thereof will be suggested to persons skilled in the art and are to be included within the spirit and purview of this application and the scope of the appended claims. In addition, any elements or limitations of any invention or embodiment thereof disclosed herein can be combined with any and/or all other elements or limitations (individually or in any combination) or any other invention or embodiment thereof disclosed herein, and all such combinations are contemplated with the scope of the invention without limitation thereto.

What is claimed:

1. A system for culturing cells or tissues embedded in a hydrogel-based construct or for applying mechanical stimuli to a hydrogel-based construct, wherein the hydrogel-based construct comprises a main body and at least one arm extending from the main body and carrying a magnetic bead at a free end of the arm, the system comprising:
a mold for forming and shaping the hydrogel-based construct, wherein the mold has a main body concave conforming the dimension of the main body of the hydrogel-based construct, and an arm concave conforming the dimension of the arm of the hydrogel-based construct; and
a magnetic attraction force-producing device configured to cyclically apply magnetic attraction force to the magnetic beads at the free end of the arm.

2. The system of claim 1, wherein the magnetic attraction force-producing device is a rail slider comprising:
a rail,
a permanent magnet disposed on the rail, and
a platform disposed on the rail for holding the hydrogel-based construct,
wherein at least one of the permanent magnets and the platform is configured to cyclically move towards the other, thereby cyclically applying magnetic attraction force to the magnetic bead at the free end of the arm.

3. The system of claim 2, wherein the rail slider further comprises a controller configured to control an operation parameter of the rail slider.

4. The system of claim 3, wherein the operation parameter of the controller configured to control the rail slider is a minimal distance between the permanent magnet and the free end of the arm when at least one of the permanent magnets and the platform moves towards the other, a moving speed of the permanent magnet and/or the platform, or a cycle period.

5. The system of claim 1, wherein the magnetic attraction force-producing device comprises an electromagnet and a platform for holding the hydrogel-based construct, and the electromagnet is configured to be cyclically activated and deactivated, thereby cyclically applying magnetic attraction force to the magnetic beads at the free end of the arm.

6. The system of claim 5, wherein the magnetic attraction force-producing device further comprises a controller configured to control an operation parameter of the force-producing device.

7. The system of claim 6, wherein the operation parameter of the controller configured to control the force-producing device is a magnetic intensity of the electromagnet when activated, a distance between the electromagnet and the free end of the arm, or an activation cycle period of the electromagnet.

8. The system of claim 1, wherein the mold is formed of a polymeric material.

9. The system of claim 8, wherein the polymeric material is polydimethylsiloxane (PDMS).

10. The system of claim 8, wherein the main body concave and/or the arm concave is formed using a soft-lithography method and/or 3D printing.

11. The system of claim 1, wherein the hydrogel-based construct is formed from a material selected from the group consisting of Gelatin Methacryloyl (GelMA), collagen, poly(ethylene glycol) diacrylate (PEGDA), methacrylated hyaluronic acid (MeHA), methacrylated chondroitin sulfate, methacrylamide chitosan (MAC), Methacrylated alginate, methacrylate and lysine functionalized dextran (Dex-MA-Ly), methacrylated gellan gum, methacrylated glycol chitosan (MeGC), Poly(ethylene oxide) (PEO), and/or poly(ethylene glycol) (PEG).

12. The system of claim 1, wherein the system is for culturing the cells, tissues, or a combination of cells and tissues embedded in the hydrogel-based construct.

13. The system of claim 12, wherein the cell or tissue is embedded in the arm of the hydrogel-based construct.

14. The system of claim 12, wherein the cell or tissue is selected from the group consisting of a chondrocyte, a tenocyte, a mesenchymal stem cell, a stem cell, a bone marrow derived stem cell (BMSC), a meniscus progenitor cell (MPC), a tendon stem cell, a stem cell derived cell, a somatic cell, a cancer cell, a muscle cell, a nerve cell, an intestinal epithelial cell, an organoid, and a tissue explant.

15. A system for applying mechanical stimuli to a hydrogel-based construct or culturing a cell or tissue embedded in a hydrogel-based construct, comprising:
a hydrogel-based construct, wherein the hydrogel-based construct comprises a main body and at least one arm extending from the main body and carrying magnetic beads at a free end of the arm; and
a magnetic attraction force-producing device configured to cyclically apply magnetic attraction force to the magnetic beads at the free end of the arm.

16. A method of testing a fatigue property of a hydrogel-based construct comprising:
i) providing the hydrogel-based construct, wherein the hydrogel-based construct comprises a main body and at least one arm extending from the main body and carrying magnetic beads at a free end of the arm;
ii) providing a magnetic attraction force-producing device configured to cyclically apply magnetic attraction force to the magnetic bead at the free end of the arm;
iii) operating the magnetic attraction force-producing device, thereby cyclically applying magnetic attraction force to the magnetic bead at the free end of the arm; and
iv) determining the fatigue property of the hydrogel-based construct by measuring tensile force, the Young's modulus, and/or mass change; identifying morphological changes of the hydrogel-based construct; and/or evaluating the hydrogel degradation from quantify the released hydrogel fragments.

17. The method of claim 16, wherein step i) comprises forming the hydrogel-based construct with a mold, wherein the mold has a main body concave conforming the dimension of the main body of the hydrogel-based construct and an arm concave conforming the dimension of the arm of the hydrogel-based construct.

18. The method of claim 17, wherein step i) further comprises:
adding a first fluidic hydrogel material to the main body concave, and optionally adding an anchoring part to the main body concave, wherein the anchoring part is configured to hold the main body in position when the magnetic attraction force is applied;
solidifying the first fluidic hydrogel material;
adding the magnetic beads to the arm concave;
adding a second fluidic hydrogel material to fix the magnetic beads; and
solidifying the second fluidic hydrogel material.

19. The method of claim 16, wherein the cyclically applied magnetic attraction force of the magnetic bead toward and away from the magnetic attraction force-producing object occurs at a frequency of about 0.1 Hz to about 10 Hz.

20. The method of claim 16, wherein the method is performed at a temperature of about 10° C. to about 50° C.

21. The method of claim 16, wherein the cyclically applied magnetic attraction force of the magnetic beads toward and away from the magnetic attraction force-producing object occurs continuously for about 5 minutes to about 24 hours per day.

22. A method of culturing cells or tissues embedded in a hydrogel-based construct, comprising:
i) providing the hydrogel-based construct, wherein the hydrogel-based construct comprises a main body and at least one arm extending from the main body, wherein the arm carries magnetic beads at a free end of the arm and the cell or tissue is embedded in the arm of the hydrogel-based construct;
ii) providing a magnetic attraction force-producing device configured to cyclically apply magnetic attraction force to the magnetic bead at the free end of the arm; and
iii) operating the magnetic attraction force-producing device, thereby cyclically applying magnetic attraction force to the magnetic bead at the free end of the arm.

23. The method of claim 22, wherein step i) comprises forming the hydrogel-based construct with a mold, wherein the mold has a main body concave conforming the dimension of the main body of the hydrogel-based construct, and an arm concave conforming the dimension of the arm of the hydrogel-based construct

24. The method of claim 23, wherein step i) comprises
adding a first fluidic hydrogel material to the main body concave, and optionally adding an anchoring part to the main body concave, wherein the anchoring part is configured to hold the main body in position when the magnetic attraction force is applied;
solidifying the first fluidic hydrogel material;
adding the magnetic beads to the arm concave;
adding a second fluidic hydrogel material to fix the magnetic beads;
solidifying the second fluidic hydrogel material;
adding a third fluidic hydrogel material comprising the cell; and
solidifying the third fluidic hydrogel material.

25. The method of claim 22, wherein the cyclically applied magnetic attraction force of the magnetic bead toward and away from the magnetic attraction force-producing object occurs at a frequency of about 0.1 Hz to about 10 Hz.

26. The method of claim 22, wherein the method is performed at a temperature of about 10° C. to about 50° C.

27. The method of claim 22, wherein the cyclically applied magnetic attraction force of the magnetic beads toward and away from the magnetic attraction force-producing object occurs continuously for about 5 minutes to about 24 hours per day.

28. The method of claim 22, wherein the embedded cells or tissues are selected from the group consisting of a chondrocyte, a tenocyte, a mesenchymal stem cell, a stem cell, a bone marrow derived stem cell (BMSC), a meniscus progenitor cell (MPC), a tendon stem cell, a stem cell derived cell, a somatic cell, a cancer cell, a muscle cell, a nerve cell, an intestinal epithelial cell, an organoid, and a tissue explant.

29. A cell or tissue culturing kit, comprising
a hydrogel-based construct, wherein the hydrogel-based construct comprises a main body and at least one arm extending from the main body and carrying a magnetic bead at a free end of the arm, and the cell or tissue is embedded in the arm of the hydrogel-based construct; and
a mold for forming and shaping the hydrogel-based construct, wherein the mold has a main body concave conforming the dimension of the main body of the hydrogel-based construct, and an arm concave conforming the dimension of the arm of the hydrogel-based construct.
